(19) United States
(12) Patent Application Publication
(10) Pub. No.: US 2014/0378932 A1
(43) Pub. Date: Dec. 25, 2014

(54) ARRAY OF DISPOSABLE ABSORBENT ARTICLES FOR FITTING BROAD RANGE OF WEARERS

(71) Applicant: The Procter & Gamble Company, Cincinnati, OH (US)

(72) Inventors: Bret Darren SEITZ, West Chester, OH (US); Gary Dean LaVON, Liberty Township, OH (US)

(21) Appl. No.: 14/309,158

(22) Filed: Jun. 19, 2014

Related U.S. Application Data
(60) Provisional application No. 61/837,416, filed on Jun. 20, 2013.

(51) Int. Cl. A61F 13/49; A61F 13/551

(52) U.S. Cl. CPC A61F 13/49011; A61F 13/55105
USPC 604/385.3

(57) ABSTRACT

An array of packages comprising three or more different sizes of absorbent articles, the array comprising first, second, and third packages comprising first, second, and third absorbent article having first, second, and third sizes. The Array Elastic Modulus of the Belt of the first, second, and third package is from about 6 gf/mm to about 15 gf/mm. And, the first, second, and third packages comprise the same brand name and sub-brand name.

### CROSS REFERENCE TO RELATED APPLICATION

This application is a continuation of, and claims priority to U.S. Patent Application Ser. No. 61/837,416, filed on Jun. 20, 2013, the entire disclosure of which is hereby incorporated by reference.

### FIELD

The present disclosure relates to an offering of an array of disposable absorbent articles sized to fit a broad range of wearers.

### BACKGROUND

Adult incontinence (“AI”) articles are designed to absorb and contain liquid and other discharges from the human body to prevent the body and clothing from becoming soiled. Adult incontinence articles come in a variety of designs, each typically available in multiple sizes. The size of the articles typically affects, for example, the size of the waist opening, the size of the openings around the thighs, and the length or “pitch” of the diaper. If a consumer selects an adult incontinence article which is size-inappropriate for the wearer, the waist opening, thigh openings or pitch of the article, for instance, may be too large for proper fit on the wearer, potentially leading to slipping, sliding, sagging, drooping, or a loss of gasketing effects that are designed to inhibit leakage. Alternatively, the waist opening, thigh opening or pitch of a size-inappropriate article may be too small for proper fit, potentially leading to wearer discomfort or skin marking of the wearer's skin.

Currently, adult incontinence articles are sold in packages that are labeled with a “size.” For example, the packages may be labeled: “Small/Medium;” “Large” or “Extra Large.” Today, AI products are marketed and sold to consumers in arrays of up to 3 sizes. This array of sizes is typically intended to cover the full range of adults. This range can span up to 300 pounds (“lbs”) (from less than 100 lbs to over 400 lbs), and also over about 40 inches in waist & hip circumference (from about 25 inches to over 64 inches). By comparison, a typical array of baby diapers is available in 7 sizes, which is intended to cover a range of only about 40 lbs and waist & hip circumferences of less than about 20 inches (preemie to toddler). Unfortunately, to date, offering an equally large number of sizes for AI products has not been considered due to incremental costs of these products and retailer shelf space. These barriers can be overcome by a compelling consumer need which the present invention delivers, which significantly improves fit, and which provides unprecedented comfort and leakage protection.

Ideally a product array should fit the broadest range of consumers with the fewest number of size offerings. Typically AI packages are labeled with a recommended wearer waist circumference range that the article is intended to fit. As a result, the waist circumference is often the sole criteria used to identify the size of an AI article. The waist size does not adequately describe the body shape of the individual and therefore does not help define the thigh circumference nor the pitch that may be needed to provide the proper fit and gasketing of the article. This is the case even though other characteristics and anthropometric attributes of potential wearers (for example, age, height, weight, thigh circumference, and rise) may vary widely within the recommended waist circumference range, and may result in an ill-fitting article even though a wearer's waist circumference falls within that range.

Further complicating the typical situation with respect to the identification and selection of size-appropriate articles is the presence of an overlap, from one “size” to the next, of the recommended waist circumference ranges on the associated packages. For example, a “Size S/M” article may have a recommended waist circumference range from about 28 to 40 inches, a “Size L” article may have a recommended waist circumference range from about 38 to 50 inches, and a “Size XL” article may have a recommended waist circumference range from about 48 to 64 inches. Such overlaps and lack of further definition of the body shape of the user may lead to ill-fitting articles.

Thus, there is a need for an array of articles which maximizes the product fit over all key body anthropometrics with the fewest number of sizes by modifying the product geometry and stretch properties of the waist and hip openings.

Offering a smaller number of sizes and still achieving proper fit, however, is challenging for many reasons, including that, obesity in the U.S. has doubled in the past 30 years. National Center for Health Statistics (NCHS) shows that greater than 35% of U.S. adults are considered obese. And, as weights get higher, other body anthropometrics get larger, though not proportionally. More of the population suffers from incontinence due to obesity, disease, and sports related issues. And, to make things even more challenging, as people age, their fit preferences change (e.g., younger women like lower fitting bikini style panties, whereas older women prefer higher fitting briefs).

Thus, it is one of the objects of the present disclosure to share how to meet the needs of the broad range of wearers with the fewest number of size offerings. As there is a relationship between: the consumer perceptions of fit; the range of consumer anthropometrics; the geometry and material properties of the article; and number of sizes available in an array, it is an object of the present disclosure to leverage waist and hip opening and elastic modulus of the belt; to achieve proper fit across the largest weight range with the fewest size offerings.

### SUMMARY

In one embodiment, an array of packages comprise a first, second, and third package. The first package comprises a first absorbent article and a first size. The second package comprises a second absorbent article and a second size. The third package comprises a third absorbent article and a third size. The Array Elastic Modulus of the Belt of the first, second, and third package is from about 6 gf/mm to about 15 gf/mm. And, the first, second, and third packages comprise the same brand name and sub-brand name.

In another embodiment, an array of packages comprise a first and a second package. The first package comprises a first absorbent article and a first size. The second package comprises a second absorbent article and a second size. The Array Elastic Modulus of the Belt of the first and second package is from about 2 gf/mm to about 5 gf/mm. And, the first and second packages comprise the same brand name and sub-brand name.

In another embodiment, an array of packages comprise a first, second, and third package. The first package comprises a first absorbent article and a first size. The second package comprises a second absorbent article and a second size. The Array Elastic Modulus of the Belt of the first, second, and third package is from about 10 gf/mm to about 15 gf/mm. And, the first, second, and third packages comprise the same brand name and sub-brand name.

In another embodiment, an array of packages comprise a first, second, and third package. The first package comprises a first absorbent article and a first size. The second package comprises a second absorbent article and a second size. The Array Elastic Modulus of the Belt of the first, second, and third package is from about 10 gf/mm to about 15 gf/mm. The Relaxed Waist Circumference for the first absorbent article is from about 30 cm to about 75 cm. The Relaxed Waist Circumference for the second absorbent article is from about 45 cm to about 90 cm. The Relaxed Waist Circumference for the third absorbent article is from about 60 cm to about 110 cm. And, the first, second, and third packages comprise the same brand name and sub-brand name.

### BRIEF DESCRIPTION OF THE DRAWINGS

FIG. 1 is a perspective view of an exemplary disposable pull-on garment in a typical in-use configuration;

FIG. 2 is a perspective view of an exemplary disposable pull-on garment in a typical in-use configuration;

FIG. 3 is a plan view of the pull-on garment in its flat uncontracted condition showing the inner surface;

FIG. 4A is a schematic cross section view of a third embodiment taken along line 4-4 in FIG. 3 of an exemplary disposable pull-on garment;

FIG. 4B is a schematic cross section view of a fourth embodiment taken along line 4-4 in FIG. 3 of an exemplary disposable pull-on garment;

FIG. 4C is a schematic cross section view of an eight embodiment taken along line 4-4 in FIG. 3 of an exemplary disposable pull-on garment;

FIG. 5A is a schematic cross section view taken along line 5-5 in FIG. 3 of an example of a folded outer leg cuff suitable in one embodiment of the invention; and

FIG. 5B is a schematic cross section view taken along line 5-5 in FIG. 3 of an alternative embodiment example of a folded leg cuff suitable in one embodiment of the invention.

FIG. 6 is an illustration of the relationship between elastic modulus of the belt and fit range.

### DETAILED DESCRIPTION

As used herein, the term “pull-on garment” refers to articles of wear which have a defined waist opening and a pair of leg openings and which are pulled onto the body of the wearer by inserting the legs into the leg openings and pulling the article up over the waist. The term “disposable” is used herein to describe garments which are not intended to be laundered or otherwise restored or reused as a garment (i.e., they are intended to be discarded after a single use and to be recycled, composted or otherwise disposed of in an environmentally compatible manner). The pull-on garment may be “absorbent” such that it absorbs and contains the various exudates discharged from the body.

As used herein, the term “absorbent article” refers to pull-on garments worn by infants and other incontinent individuals, including adults, to absorb and contain urine, feces and/or menses. It should be understood, however, that the term absorbent article is also applicable to other garments such as training pants, incontinent briefs, feminine hygiene garments or panties, and the like.

As used herein, the term “belt” may include a waistband, ears, side-panels, back panels, etc.

As used herein, the terms “elastic,” “elastomer,” and “elastomeric” refer to a material which generally is able to extend to a strain of at least 50% without breaking or rupturing, and is able to recover substantially to its original dimensions, accounting for set, after the deforming force has been removed.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

As used herein, the term “array” means a display of packages comprising disposable articles of different sizes having like article constructions (e.g., same elastomeric materials [compositionally and/or structurally] in the flaps, graphic elements) said packages having the same brand and/or sub-brand, and said packages oriented in proximity to each other in a given area of a retail store. An array is marketed as a line-up of products normally having like packaging elements (e.g., packaging material type, film, paper, dominant color, design theme, etc.) that convey to consumers that the different individual packages are part of a larger line-up. Arrays often have the same brand, for example, “Depend,” and same sub-brand, for example, “for Women Underwear.” A different array may have the brand “Depend” and the sub-brand “Silhouette For Women.” The differences between the “for Women Underwear” array and the “Silhouette For Women” arrays include different elastomeric materials in the side flaps, where “for Women Underwear” comprises strands as the elastomeric material and “Silhouette For Women” comprises a film elastomeric material.” Furthermore, the packaging is distinctly different in that “for Women Underwear” is packaged in a predominately green, film bag and “Silhouette For Women” is packaged in a predominately maroon box.

Further regarding “arrays,” as another example of two separate “arrays” having the same brand, “Certainty,” one line-up has the sub-brand “Women's Underwear.” A different array may have the same brand “Certainty” and the sub-brand “Smooth Shape Briefs for Women.” The differences between the “Women's Underwear” array and the “Smooth Shape Briefs for Women” arrays include different elastomeric materials in the side flaps, where “Women's Underwear” comprises strands as the elastomeric material and “Smooth Shape Briefs for Women” comprises a film elastomeric material.” Furthermore, the packaging is distinctly different in that “Women's Underwear” is packaged in a predominately blue, film bag and “Smooth Shape Briefs for Women” is packaged in a predominately maroon box.

Arrays also often have the same trademarks, including trademarks of the brand, sub-brand, and/or features and/or benefits across the line-up.

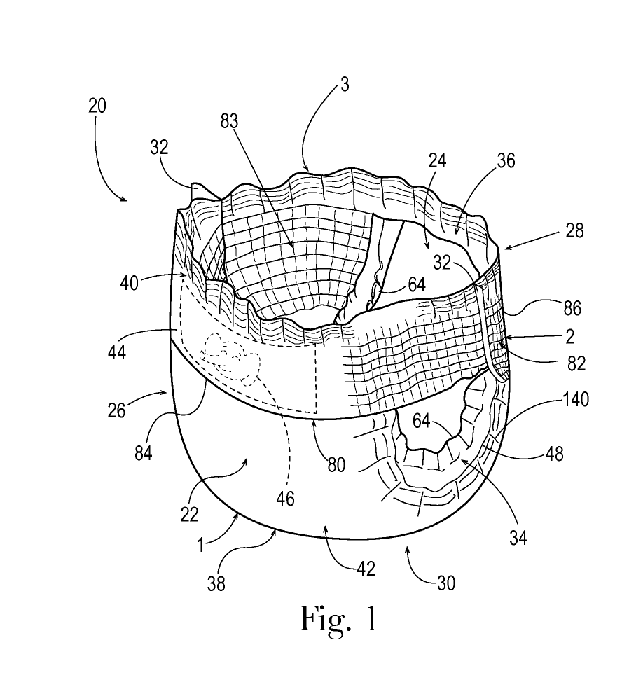

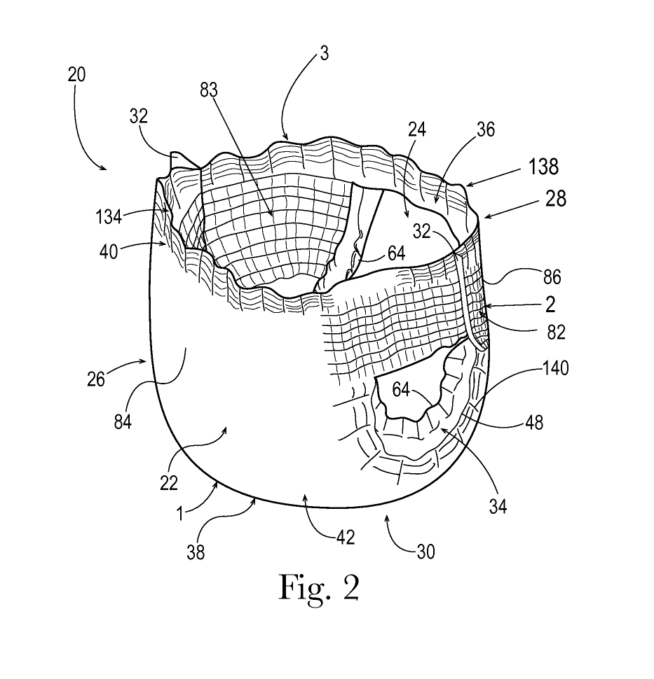

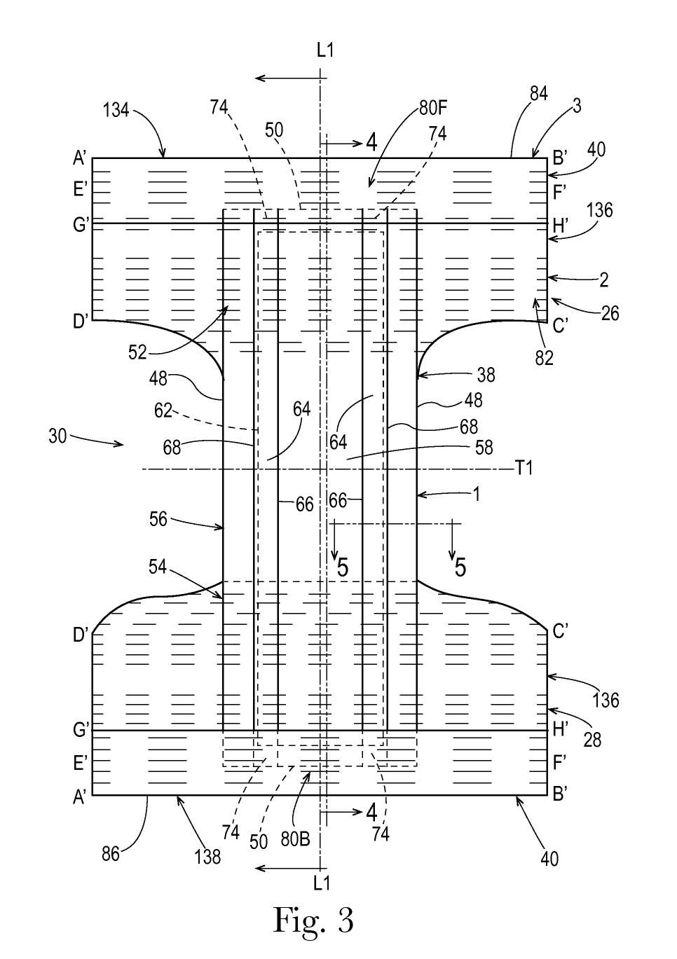

As used herein, the term “on-line array” means an “array” distributed by a common on-line source. FIGS. 1 and 2 are perspective views of the absorbent article 20. As shown in FIG. 3, the absorbent article 20 has a longitudinal centerline L1 and a transverse centerline T1. The absorbent article 20 has an outer surface 22, an inner surface 24 opposed to the outer surface 22, a front region 26, a back region 28, a crotch region 30, and seams 32 which join the front region 26 and the back region 28 to form two leg openings 34 and a waist opening 36. Also referring to FIGS. 1-3, the absorbent article 20 comprises a main portion 1, a side portion 2, and a waist portion 3.

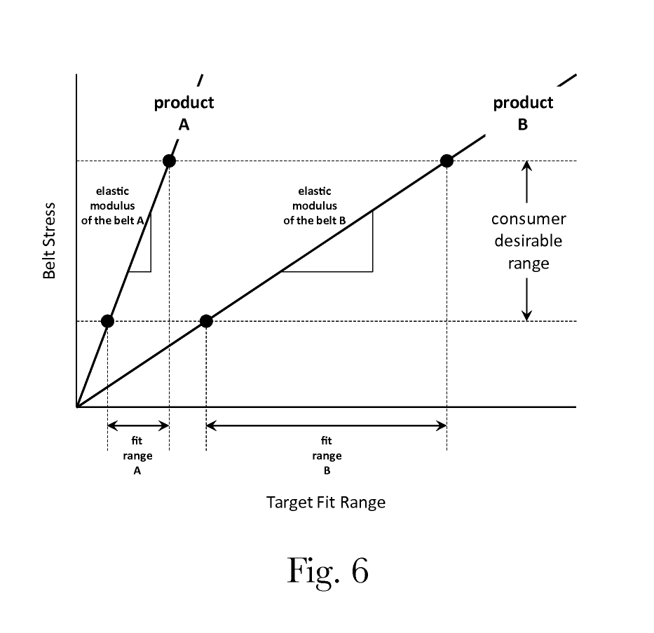

In the embodiment shown in FIGS. 1 and 3, the absorbent article 20 comprises an absorbent main body 38 (hereinafter may be referred to as “main body” or “central chassis”) to cover the crotch region of the wearer and a belt 40 extending transversely about the waist opening 36. The absorbent article 20 may also comprise an outer cover layer 42 to cover the main body 38. The belt 40 defines the waist opening 36. The belt 40, the main body 38 and/or the outer cover layer 42 jointly define the leg opening 34. As shown in FIG. 3 and FIG. 6, the portions of the belts making up leg openings 34 may be shaped. While FIG. 3 illustrates discrete first belt layers 83, FIG. 6 illustrates a full outer cover nonwoven first belt layer 82.

In the embodiment shown in FIG. 2, the absorbent article 20 comprises an absorbent main body 38 to cover the crotch region of the wearer and a belt 40 extending transversely about the waist opening 36. The absorbent article 20 may also comprise an outer cover layer 42 to cover the main body 38. The belt 40 defines the waist opening 36. The belt 40, the main body 38 and/or the outer cover layer 42 jointly define the leg opening 34. One or more of the belt layers may extend from a first waist edge 134 in a first waist region 26 through the crotch region to a longitudinally opposing second waist edge 138 in a second waist region 28 and may form a portion or the whole of the outer surface of the absorbent article 20.

The absorbent main body 38 absorbs and contains body exudates disposed on the main body 38. In the embodiment shown in FIG. 3, the main body 38 has a generally rectangular shape having a longitudinal centerline L1, a transverse centerline T1, left and right longitudinally extending side edges 48 (hereinafter may be referred to as “longitudinal side edge”) and front and back transversely extending end edges 50 (hereinafter may be referred to as “transverse end edge”). The main body 38 also has waist panels (i.e., a front waist panel 52 positioned in the front waist region 26 of the absorbent article 20 and a back waist panel 54 positioned in the back waist region 28) and a crotch panel 56 in the crotch region 30 between the front and back waist panels 52, 54.

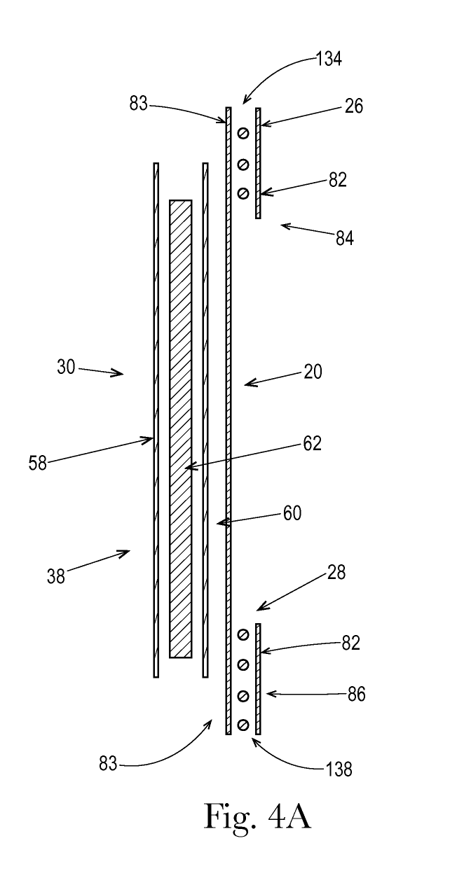

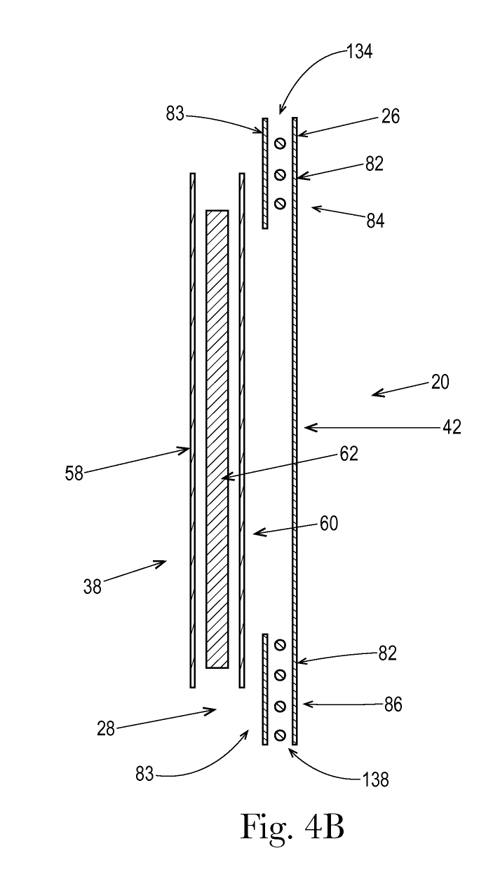

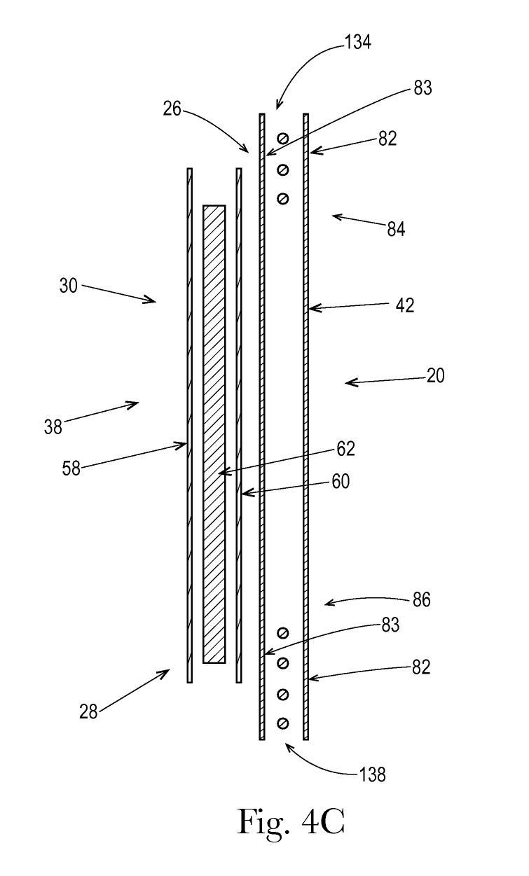

In the embodiment shown in FIGS. 4A and 4B, the absorbent articles 20 may comprise front and rear extensible belts 84, 86 disposed in the front and rear waist regions 26, 28 respectively and intended to encircle at least a portion of the waist of the wearer, the front and rear belts 84, 86 being connected by the main body that forms the crotch region 30 of the article. The first and second belt may be formed from a first belt layer 82 (e.g., a “full outer cover nonwoven” or an “outer cover web”) extending from a first waist edge 134 in a first waist region 26 through the crotch region to a longitudinally opposing second waist edge 138 in a second waist region 28 and forming a portion of the outer surface of the absorbent article 20. The front and rear belts 84, 86 also may comprise a second belt layer 83a and b (e.g., an “inner belt web”) forming a portion of the inner surface 24 of the absorbent article, the second belt layer may be formed of two longitudinally spaced webs of material. The first and second belt portions may also comprise an elastomeric material 200 (e.g., “elastic elements” or “elastics”) disposed between the first and second belt layers. The elastomeric material may comprise elastic strands, elastomeric films, elastomeric ribbons, elastomeric nonwovens, elastomeric filaments, elastomeric adhesives, elastomeric foams, scrims or combinations thereof. The main body 38 (“central or center chassis”) of the absorbent article may comprise an outer surface 22, backsheet 60, an inner surface 24, topsheet 58, and an absorbent core 62 disposed between the topsheet 58 and the backsheet 60. The first belt layer may form a portion of the outer surface 22. In addition, the main body may comprise elasticized barrier leg cuffs 64 disposed at or adjacent the side edges of the main body. The second belt layer may overlap at least a portion of the main body and one or both of the second belt layer webs may form the outer surface of the first belt layer or alternatively the inner surface of the first belt layer. Alternatively, the front portion and/or the rear portion of the first belt layer 82 may be folded along the waist edge of the belt region to wrap the elastomeric material and form a portion of the second belt layer of one or both of the front and rear belt portions 84, 86. In other words, the inner surface and outer surface of each of the belt portions may be formed from a single web of material.

In the embodiment shown in FIG. 4C, the absorbent articles 20 may comprise front and rear belts 84, 86 intended to encircle at least a portion of the waist of the wearer, the front and rear belts 84, 86 being connected to a main body 38 forming a portion of the crotch region 30 of the absorbent article 20. The front and rear belts 84, 86 are formed from a first belt layer 82 forming a portion of the outer surface of the absorbent article. The front and rear belt portions 84, 86 also comprise a second belt layer 83 forming a portion of the inner surface 24 of the absorbent article 20. The second belt layer may be laterally discontinuous and spaced apart in a transverse direction. The first and second belt layers 82, 83 may be formed of substantially the same material or may comprise different materials. The first and second belt layers 82, 83 may be formed from nonwovens, films, foams or combinations thereof. The front and rear belt portions 84, 86 may also comprise an elastomeric material disposed between the first and second belt layers 82, 83. The elastomeric material may comprise elastic strands, elastomeric films, elastomeric ribbons, elastomeric nonwovens, elastomeric filaments, elastomeric adhesives, elastomeric foams, scrims or combinations thereof. A portion of the elastomeric material may be directly combined with the outer cover layer. The main body 38 of the absorbent article may comprise an outer surface 22, backsheet 60, an inner surface 24, topsheet 58, and an absorbent core 62 disposed between the topsheet 58 and the backsheet 60. In certain embodiments the backsheet may be a nonwoven and film laminate wherein the nonwoven is formed by the outer cover layer. In addition, the main body 38 may comprise elasticized barrier leg cuffs 64 disposed at or adjacent the side edges of the main body 38. The front and rear belts 84, 86 overlap at least a portion of the main body 38 and one or both of the belts may be disposed on the outer surface of the main body 38. A portion of the second belt layer and/or a portion of the first belt layer may be directly attached to the outer cover layer. The front and rear belts 84, 86 may be formed from a first belt layer extending from a first waist edge 134 in a first waist region 26 through the crotch region to a second waist edge 138 in a second waist region 28 and forming a portion of the outer surface of the absorbent article 20. The front and rear belts 84, 86 may also comprise a second belt layer extending from a first waist edge 134 in a first waist region 26 through the crotch region to a second waist edge 138 in a second waist region 28 and forming a portion of the inner surface of the absorbent article 20. The first and second belt layers may be formed of substantially the same material or may comprise different materials. The first and second belt layers may be formed from nonwovens, films, foams, woven materials or combinations thereof. The front and rear belt portions 84, 86 may also comprise an elastomeric material disposed between the first and second belt layers in one or both of the first and second waist regions 26, 28. The elastomeric material may comprise elastic strands, elastomeric films, elastomeric ribbons, elastomeric nonwovens, elastomeric filaments, elastomeric adhesives, elastomeric foams, scrims or combinations thereof. The main body 38 of the absorbent article 20 may comprise an outer surface 22, backsheet 60, an inner surface 24, topsheet 58, and an absorbent core 62 disposed between the topsheet 58 and the backsheet 60. One or both of the first and second belt layers may form a portion of the outer surface 22. In addition, the main body 38 may comprise elasticized barrier leg cuffs 64 disposed at or adjacent the side edges of the main body 38. A portion of one or both of the front and rear belts 84, 86 may overlap at least a portion of the main body 38. Alternatively, the front belt portion and rear belts 84, 86 may comprise a belt layer forming a first surface of the belt portion wherein the belt layer may be folded along the waist edge of the belt portion to wrap the elastomeric material and overlap a portion of the opposing belt layer. In other words, a portion of the inner surface and a portion of the outer surface of each of the belt portions may be formed from a single web of material.

Additional lateral extensibility in the main body 38 may be provided in a variety of ways. For example, a material or materials from which the main body 38 is made may be pleated by any of many known methods. Alternatively, all or a portion of the main body 38 may be made of a formed web material or a formed laminate of web materials like those described in U.S. Pat. No. 5,518,801 issued on 21 May 1996 in the name of Chappell et al. This formed web material includes distinct laterally extending regions in which the original material has been altered by embossing or another method of deformation to create a pattern of generally longitudinally oriented alternating ridges and valleys and also includes laterally extending unaltered regions between the laterally extending altered regions. The formed web material can be extended in a direction perpendicular to the ridges up to the point where the ridges and valleys flatten with substantially less force than is required to extend beyond that point. In addition to lateral extensibility, the creation of a formed laminate web as described above provides a main body 38 backsheet with improved texture and cloth-like appearance and feel. The deformation creates a cloth-like pattern in the film and increases the loft of the nonwoven in multi-layer film and nonwoven laminate backsheets.

Alternatively, a portion of the absorbent article can be ring-rolled and thus rendered highly extensible as described in U.S. Pat. No. 5,366,782 (issued Nov. 22, 1994 to Curro, et al). Specifically, a ring-rolling apparatus includes opposing rolls having intermeshing teeth that incrementally stretch and thereby plastically deform the material forming the absorbent article (or a portion thereof) thereby rendering the article extensible in the ring-rolled regions. In one embodiment, the absorbent article can be ring-rolled in a portion of at least one of the front or back waist regions, for example the portion of the main body 38 underlying and/or immediately adjacent one or both of the front and back belts 84, 86, while other regions may comprise a structured elastic-like formed web material. The article may be ring-rolled across the entire width in one or both of the waist regions or alternatively may be ring-rolled over only a portion of the main body 38 width or over only a portion of one or both of the belts.

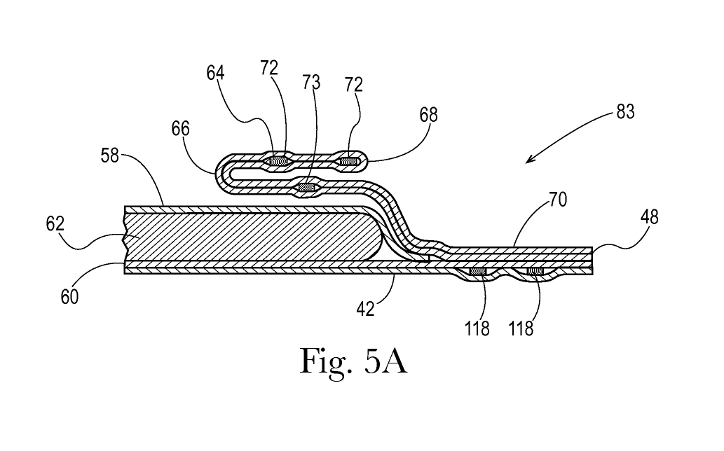

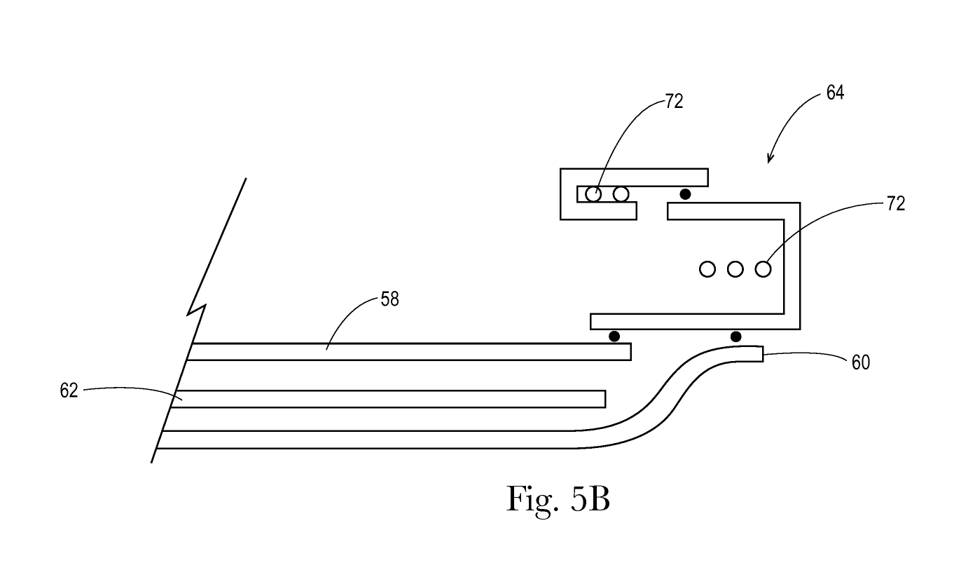

The main body 38 may comprise a liquid pervious topsheet 58, a liquid impervious backsheet 60 and an absorbent core 62 disposed therebetween. The main body 38 may additionally comprise a barrier leg cuff 64 disposed along the longitudinal side edge 48. The barrier leg cuff 64 provides improved containment of liquids and other body exudates in the crotch region 30. The barrier leg cuff 64 shown in FIG. 5 comprises a single layer of material which may be folded to form a barrier leg cuff having two layers. The barrier leg cuff 64 extends from the side of the main body at or adjacent the longitudinal side edge 48 toward the longitudinal centerline L2. The barrier leg cuff may be folded along the folding line 66 back toward the longitudinal side edge 48. The barrier leg cuff 64 may have a first barrier cuff elastic material 72 adjacent to the distal portion 68 and a second barrier cuff elastic material 73 adjacent to the proximal portion 70 of the barrier leg cuff 64. The proximal portion 70 of the barrier leg cuff 64 may be joined to the backsheet 6o adjacent to the longitudinal side edge 48. The portion of the barrier leg cuff 64 along the folding line 66 and the distal portion 68 may be free from attachment to any portion of the main body 38 in the crotch region 30 such that the barrier leg cuff 64 stands up toward the wearer's body. The transverse end 74 of the barrier leg cuff 64 may be joined to the topsheet 58 at or adjacent the longitudinally opposing ends of the leg cuff by an attachment means which may be any known means such as an adhesive, heat bond, pressure bond or the like as shown in FIG. 5A. Examples of acceptable cuffs 64 are disclosed in U.S. Ser. No. 13/457,521, filed Apr. 27, 2012, including the configurations disclosed by FIGS. 8a-t. For instance, as illustrated in FIG. 5B, the barrier leg cuff may be a two-piece cuff. And, the cuff 64 may be joined to the backsheet with a no leak bead 215 that runs along the entire longitudinal length of the cuff and/or the backsheet film 60.

The liquid pervious topsheet 58 may be positioned adjacent the body-facing surface of the absorbent core 62 and may be joined thereto and/or to the backsheet 60 by any attachment means known in the art. The liquid impervious backsheet 60 is generally that portion of the absorbent article 20 positioned adjacent the garment-facing surface of the absorbent core 62 and prevents the exudates absorbed and contained therein from soiling articles that may contact the absorbent article 20. The absorbent core is positioned between the topsheet 58 and the backsheet 60 and absorbs and retains liquids such as urine and other certain body exudates.

The topsheet 58, the backsheet 60 and the absorbent core may be manufactured with any known materials. Suitable topsheet materials may include porous foams; reticulated foams; apertured plastic films; or woven or nonwoven webs of natural fibers (e.g., wood or cotton fibers), synthetic fibers (e.g., polyester or polypropylene fibers), or a combination of natural and synthetic fibers. Suitable backsheet materials may include breathable materials that permit vapors to escape from the diaper while still preventing exudates from passing through the backsheet.

A suitable absorbent core for use in the absorbent article 20 may comprise any absorbent material (such as absorbent gelling material or super absorbent polymer) which is generally compressible, conformable, non-irritating to the wearer's skin, and capable of absorbing and retaining liquids such as urine and other certain body exudates. In addition, the configuration and construction of the absorbent core may also be varied (e.g., the absorbent core(s) or other absorbent structure(s) may have varying caliper zones, hydrophilic gradient(s), a superabsorbent gradient(s), or lower average density and lower average basis weight acquisition zones; or may comprise one or more layers or structures). In some embodiments, the absorbent core may comprise a fluid acquisition component, a fluid distribution component, and a fluid storage component. An example of a suitable absorbent core having a fluid acquisition component, a fluid distribution component, and a fluid storage component is described in U.S. Pat. No. 6,590,136. Examples of acceptable air felt free cores (i.e., absorbent core systems having little or no air felt) are disclosed in U.S. Pat. Nos. 5,562,646, 7,750,203, 7,744,576 and U.S. Pub. Nos. 2008/0312617A1, 2008/0312619A1, and 2004/0097895A1.

The outer cover layer 42 may be disposed on the outer surface 22 of the absorbent article 20 and covers the crotch panel 56 of the absorbent main body 38. The outer cover layer 42 may extend into and cover the front waist panel 52 and the back waist panel 54 of the main body 38. The outer cover layer may form a portion of the backsheet and/or the main body. The outer cover layer 42 may be directly joined to and cover a portion or all of the liquid impervious backsheet 60 of the main body 38. The central panel 80 of the front and back belt 84, 86, forming respectively front and rear central panels 80F, 80B may be joined to the front waist panel 52 and the back waist panel 54 of the main body 38 through the outer cover layer 42. Thus, the outer cover layer 42 is disposed between the front and back belt 84, 86 and the liquid impervious backsheet 60 of the main body 38. In one embodiment shown in FIGS. 2 and 4A, the outer cover layer 42 is coextensive with the liquid impervious backsheet 60. The leg elastic material 140 is disposed so as to extend generally longitudinally along the longitudinal side edge 48 of the main body 38. The leg elastic material 140 may be disposed at least in the crotch region 30 of the absorbent article 20 or may be disposed along the entirety of the longitudinal side edge 48.

The outer cover layer 42 may comprise a material separate from the material of the inner layer 83 and the outer layer 82 constituting the belt 40. The outer cover layer 42 may comprise two or more layers of materials. The outer cover layer 42 may comprise any known materials and may comprise materials used for the front and back belt 84, 86 as explained above. The outer cover layer 42 may comprise a single layer of nonwoven web of synthetic fibers. The outer cover layer 42 may comprise a single layer of hydrophobic, non-stretchable nonwoven material. The outer cover layer may comprise a film, a foam, a nonwoven, a woven material or the like and/or combinations thereof such as a laminate of a film and a nonwoven.

The belt 40 may comprise a front belt 84 and a back belt 86 and has a ring-like configuration by permanently or refastenably connecting the front belt 84 and the back belt 86 at the seams 32 or by permanently or refastenably connecting the front and/or back belt to the main body 38. Articles of the present disclosure may have refastenable elements, configurations, and methods of making as disclosed in U.S. Ser. No. 61/787,416, filed on Mar. 15, 2013, as well as U.S. Ser. No. 61/787,332, filed on Mar. 15, 2013. The refastenable elements may be fastened during the manufacturing process and/or fastened in the package prior to use by the wearer or caregiver (i.e., the pant may be sold in “closed form”). The front and back belts 84 and 86 may be in a number of configurations as described and illustrated in FIGS. 3A-C and 4A-k of U.S. Ser. No. 61/666,065, filed on Jun. 29, 2012, titled DISPOSABLE ABSORBENT REFASTENABLE PANTS AND METHODS FOR MANUFACTURING THE SAME. Further, the absorbent articles of this disclosure may be manufactured in accordance with the descriptions and illustrations of U.S. Ser. No. 61/666,065 (see, for example, FIGS. 5-10C of U.S. Ser. No. 61/666,065).

The belt 40 may be ring-like and elastic. The ring-like elastic belt 40 extends transversely about the waist opening 36 of the absorbent article 20 and acts to dynamically create fitment forces and to distribute the forces dynamically generated during wear. Applicants have found that improved fit can be created by controlling the linear density of, and the pre-strain of, and the distance between the elastomeric material in relation to each other and to the openings for the body. This may occur by choosing different materials throughout the belt 40 that exhibit desired properties. The different materials are combined at specific distances, linear densities, and prestrains to create a belt 40 that acts to dynamically create fitment forces. Particularly, the articles of this disclosure may have the characteristics of the articles of Examples 1-4 as disclosed in U.S. Ser. No. 13/764,990, filed Feb. 12, 2013. Articles of the present disclosure may also have the same stress, strain and spacing of its elastics as disclosed in U.S. Ser. No. 13/764,990 and/or as disclosed in U.S. Ser. No. 61/598,012, filed Feb. 13, 2012. Articles of the present disclosure may also have the same elastic sections and force zones disclosed in U.S. Ser. No. 13/764,990.

The front belt 84 may comprise 5 to 50 elastic strands. The front belt 84 may comprise 10 to 20 elastic strands. The back belt 86 may comprise 5 to 50 elastic strands. The back belt 86 may comprise 10 to 20 elastic strands. The elastic strands are distributed amongst the different force zones. Elastic strands may be distributed evenly amongst the force zones. Elastic strands may also be distributed unevenly amongst the different force zones. Each force zone comprises at least one elastic strand.

The elastic strands may have a linear density between 200 to 2500. Linear density is the density of the elastic fibers in the elastic strand. The most commonly used unit for the linear density is the decitex, abbreviated “dtex,” which is the mass in grams per 10,000 meters. The linear density may be used to change the force profile. For example, one could reach a desired force profile by selecting the linear density of a single elastic strand, combining multiple elastic strands with a smaller linear density in close proximity to each other, and/or combining with other elastomeric materials.

The elastic strands may have an elastic pre-strain. The elastic pre-strain is the percent of length increase in an elastic strand or plurality of elastic strands at the point of combining the elastic(s) with the first and/or second belt layers. For example a strand with a free length of 15 centimeters (cm) may have a load applied such that the 15 cm elastic strand is now 18 cm long. This length increase of 3 cm is 20% of 15 cm (3/15), or a 20% strain. The elastic pre-strain may be used to change the force profile of a single elastic strand or a plurality of elastic strands. Force profiles may also be changed by changing the linear density in conjunction with the elastic pre-strain of one or more elastic strands.

The number of elastic strands in each zone may be changed according to the placement of the absorbent core. Applicants have found that the use of thinner absorbent cores may lead to a need in increased elastic force to compensate for the change in article thickness. The force profile must be adjusted depending on the location and thickness of the absorbent core. This particularly affects the second elastic section and third elastic section.

The elastic strands disposed in the belt may be aligned in a curved fashion so that a tangent of the curve of the elastic strands may form an acute angle with the centerline or may form an arcuate shape. This may allow for targeting the force profile and/or coordinating print and elastication/rugosities/elastics in the stretch sections.

The elasticized belts and rear region of the pant may be in a number of configurations as described and illustrated in FIGS. 3A-C and 4A-k of U.S. Ser. No. 61/666,065, filed on Jun. 29, 2012, titled DISPOSABLE ABSORBENT REFASTENABLE PANTS AND METHODS FOR MANUFACTURING THE SAME. Further, the absorbent articles of this disclosure may be manufactured in accordance with the descriptions and illustrations of U.S. Ser. No. 61/666,065 (see, for example, FIGS. 5-10C of U.S. Ser. No. 61/666,065).

It may be desirable to use the hot air seaming processes, as well as the article forming processes disclosed in U.S. Pat. No. 6,248,195 and U.S. Ser. No. 12/795,021, 13/401,907, and Ser. No. 13/402,056 for seaming articles as disclosed herein. And, the articles disclosed herein may have graphics (e.g., 46) in accordance with U.S. Ser. Nos. 61/646,953 and 61/646,979, each filed on May 15, 2012.

It may be desirable to link the number of sizes in an array to the Elastic Modulus of the Belt. The higher the Elastic Modulus of the Belt, the more sizes may need to be offered to achieve proper fit given the range over which consumers perceive the product to be comfortable. This is illustrated in FIG. 6. Shown are two products with different Elastic Modulus of the Belt. As product A has a higher Elastic Modulus of the Belt than does product B, the fit range over which it delivers a given belt stress range is smaller than that of product B. Hence, in order to cover the target fit range, more packages of product A would be needed than with product B.

The full range of waist and hip circumference for adults (99% of the population) spans from approximately 24 inches (60 cm) to 64 inches (163 cm). A desirable range of Elastic Modulus of the Belt may be from about 2 gf/mm to about 20 gf/mm, or from about 2 gf/mm to about 15 gf/mm, or from about 3 gf/mm to about 12 gf/mm, or from 3 gf/mm to about 7 gf/mm. When the Elastic Modulus of the belt falls below 2 gf/mm, the product may “feel” loose with little elastic recovery. And, when the Elastic Modulus of the belt gets above the upper portion of the range at 20 gf/mm, the product may “feel” tight and un-stretchable. A consumer desirable range of belt stress (the pressure the belt imparts to the consumer's body) may be from about 2 gf/mm to about 9 gf/mm, or from about 3 gf/mm to about 7 gf/mm. Belt stresses (pressure on body) above 9 gf/mm feel tight and uncomfortable and can potentially lead to skin marking. Belt stresses (pressure on body) below 2 gf/mm feel loose, and may not sustain fit or gasketing.

As an array of sizes should fit waist (i.e., waist circumference [omphalion]− the horizontal circumference of the waist at the level of the center of the navel [omphalion]) and hip (i.e., the circumference of the body measured at the level of the maximum posterior protuberance of the buttocks) circumferences from 60 cm up to 163 cm, there exists a correlation between the required number of sizes in the array, the Relaxed Waist Circumference of each product in that array, and the Array Elastic Modulus of the Belt material for that array. Thus it is possible to determine the maximum allowable Array Elastic Modulus of the Belt for any number of sizes offered in an array. More importantly, it enables the determination of the exact number of sizes required in an array necessary to fit the targeted range for any given array modulus of the belt. This is shown below in Table 1.

TABLE 1
Maximum Allowable Array Elastic Modulus of Belt for the Number of Sizes in an Array

| | Number of Sizes in an Array | | | | |
|---|---|---|---|---|---|
| | 2 | 3 | 4 | 5 | 6 |
| | Maximum Allowable Array Elastic Modulus of Belt (gf/mm) | | | | |
| | 3.2 | 7.1 | 11.1 | 15.1 | 19.1 |
| Array Size | waist opening: 31 | waist opening: 42 | waist opening: 47 | waist opening: 50 | waist opening: 52 |
| 1 (cm) | waist range: 60-99 | waist range: 60-84 | waist range: 60-77 | waist range: 60-73 | waist range: 60-71 |
| Array Size | waist opening: 51 | waist opening: 59 | waist opening: 61 | waist opening: 61 | waist opening: 61 |
| 2 (cm) | waist range: 99-163 | waist range: 84-117 | waist range: 77-99 | waist range: 73-90 | waist range: 71-84 |
| Array Size | n/a | waist opening: 82 | waist opening: 78 | waist opening: 75 | waist opening: 72 |
| 3 (cm) | | waist range: 117-163 | waist range: 99-127 | waist range: 90-109 | waist range: 84-99 |
| Array Size | n/a | n/a | waist opening: 100 | waist opening: 91 | waist opening: 86 |
| 4 (cm) | | | waist range: 127-163 | waist range: 109-134 | waist range: 99-117 |
| Array Size | n/a | n/a | n/a | waist opening: 111 | waist opening: 101 |
| 5 (cm) | | | | waist range: 134-163 | waist range: 117-138 |
| Array Size | n/a | n/a | n/a | n/a | waist opening: 119 |
| 6 (cm) | | | | | waist range: 138-163 |

For example, if the Array Elastic Modulus of the Belt is greater than 7.1 gf/mm, then the array may require more than 3 sizes in order to fully fit the range. For a 3 size array, it may be desired to have the Array Elastic Modulus of the Belt less than 7.1 gf/mm to allow for some overlap between the sizes.

To further illustrate the linkage between the number of sizes in an array to the Array Elastic Modulus of the Belt, Table 2 below shows the implications of eliminating just one size from the number of sizes required by the disclosed inventive arrays (at the maximum allowable Array Elastic Modulus of the Belt). For example, with an Array Elastic Modulus of the Belt of 7.1 gf/mm, the inventive number of sizes required is 3. This ensures coverage of 100% of the target range (60 cm-163 cm). If instead, a product array of 2 sizes was offered with an Array Elastic Modulus of the Belt of 7.1 gf/mm, then the product array could only fit up to 77% of the targeted range.

TABLE 2
Percent of Fit Range Covered due to Reduction of One
Size at Max Allowable Array Elastic Modulus of Belt

| | Array Elastic Modulus of Belt (gf/mm) | | | | |
|---|---|---|---|---|---|
| | 3.2 | 7.1 | 11.1 | 15.1 | 19.1 |
| Inventive Number of Sizes | 2 | 3 | 4 | 5 | 6 |
| Required for Given Array | | | | | |
| Elastic Modulus | | | | | |
| Percent of Range | 100% | 100% | 100% | 100% | 100% |
| (60 cm-163 cm) | | | | | |
| Covered | | | | | |
| Inventive Number of Sizes | 1 | 2 | 3 | 4 | 5 |
| Required minus 1 | | | | | |
| Maximum Possible Percent | 62% | 77% | 83% | 87% | 89% |
| of Range (60 cm-163 cm) | | | | | |
| now Covered | | | | | |

The relationship between the number of packages offered in an array, the Relaxed Waist Circumference, Elastic Modulus of the Belt for each package in that array, and the range over which that array fits is shown in Table 3 for some marketed product arrays.

TABLE 3
Examples of Existing Product Arrays

| | Assurance | Depend for | Depend |
|---|---|---|---|
| | Underwear | Women | Silhouette for |
| | for Women | Underwear | Women Briefs |
| Product | (Maximum | (Maximum | (Maximum |
| Array | Absorbency) | Absorbency) | Absorbency) |
| Number of Packages | 3 | 3 | 2 |
| in Array | | | |
| First Package | S/M | S/M | S/M |
| in Array | | | |
| First Package | 46 | 50 | 71 |
| Relaxed Waist | | | |
| Circumference (cm) | | | |
| First Package | 4.9 | 5.4 | 12.9 |
| Elastic Modulus of | | | |
| the Belt (gf/mm) | | | |
| First Package | 74-112 | 78-115 | 88-110 |
| Approximate | | | |
| Range From 3 | | | |
| to 7 gf/mm (cm) | | | |
| Second Package | L | L | L/XL |
| in Array | | | |
| Second Package | 53 | 53 | 81 |
| Relaxed Waist | | | |
| Circumference (cm) | | | |
| Second Package | 5.3 | 5.8 | 11.3 |
| Elastic Modulus of | | | |
| the Belt (gf/mm) | | | |
| Second Package | 83-123 | 80-117 | 103-131 |
| Approximate | | | |
| Range From 3 | | | |
| to 7 gf/mm (cm) | | | |
| Third Package | XL | XL | na |
| in Array | | | |
| Third Package | 63 | 67 | na |
| Relaxed Waist | | | |
| Circumference (cm) | | | |
| Third Package | 5.0 | 5.0 | na |
| Elastic Modulus of | | | |
| the Belt (gf/mm) | | | |
| Third Package | 101-151 | 107-161 | na |
| Approximate | | | |
| Range From 3 | | | |
| to 7 gf/mm (cm) | | | |
| Array Elastic | 5.1 | 5.4 | 12.1 |
| Modulus of | | | |
| the Belt (gf/mm) | | | |
| Array Approximate | 74-151 | 78-161 | 88-131 |
| Range From 3 | | | |
| to 7 gf/mm (cm) | | | |
| Array Percent | 75% | 81% | 42% |
| Within Target | | | |
| Range of 60-163 cm | | | |

It can be seen that the product array Depend Silhouette for Women Briefs covers less of the target range of 60 cm to 163 cm while delivering between 3 gf/mm to 7 gf/mm than the other listed arrays. This is due to the higher Array Elastic Modulus of the Belt and fewer packages than the other two listed arrays.

Table 4 below illustrates inventive arrays (Array Elastic Modulus of the Belt and Relaxed Product Circumferences) for array sizes of 2, 3 and 4 packages for covering the target range of 60 cm to 163 cm while delivering between 3 gf/mm to 7 gf/mm.

TABLE 4
Inventive Arrays - Maximizes the Percent Fit Within
the Target Range while Minimizing the number of Sizes

| Product | Array of 2 | Array of 3 | Array of 4 |
|---|---|---|---|
| Array | Packages | Packages | Packages |
| First Package in Array | Size 1 | Size 1 | Size 1 |
| First Package Relaxed | 25-37 | 34-50 | 38-56 |
| Waist Circumference (cm) | | | |
| First Package Elastic | <=3.2 | <=7.1 | <=11.1 |
| Modulus of the | | | |
| Belt (gf/mm) | | | |
| Second Package in Array | Size 2 | Size 2 | Size 2 |
| Second Package Relaxed | 41-61 | 47-71 | 49-73 |
| Waist Circumference (cm) | | | |
| Second Package Elastic | <=3.2 | <=7.1 | <=11.1 |
| Modulus of the | | | |
| Belt (gf/mm) | | | |
| Third Package in Array | na | Size 3 | Size 3 |
| Third Package Relaxed | na | 66-98 | 62-94 |
| Waist Circumference (cm) | | | |
| Third Package Elastic | na | <=7.1 | <=11.1 |
| Modulus of the | | | |
| Belt (gf/mm) | | | |
| Fourth Package in Array | na | na | Size 4 |
| Fourth Package Relaxed | na | na | 80-120 |
| Waist Circumference (cm) | | | |
| Fourth Package Elastic | na | na | <=11.1 |
| Modulus of the | | | |
| Belt (gf/mm) | | | |
| Array Elastic | <=3.2 | <=7.1 | <=11.1 |
| Modulus of the | | | |
| Belt (gf/mm) | | | |
| Array Percent Fit | 100% | 100% | 100% |
| Within Target | | | |
| Range of 60-163 cm | | | |

Absorbent articles as disclosed herein may be manufactured by the same company on the same manufacturing line and may be sold in an array under the same brand (e.g., Pampers, Huggies, Depends, Always) and/or sub-brand name (Cruisers, Swaddlers, and Easy Ups, Baby Dry, Silhouette, etc.).

Identical or Substantially Identical Chassis

As disclosed in U.S. Pub. No. 2013-0211355, it may be desirable to offer an array of packages for fitting different sized wearers, but comprising identical or substantially identical chassis. For instance, an array may comprise a first package comprising a first size of absorbent articles and a second package may comprise a second size of absorbent articles, where the first and second packages comprise identical or substantially identical chassis as described in U.S. Pub. No. 2013-0211355. More particularly, the first package may comprise a first chassis and the second package may comprise a second chassis, where each of the first and second chassis comprise the same dimensions of one or more of: core width at the lateral centerline, core width at one of the front or rear core end, a distance from a left outer cuff distal edge to a right outer cuff distal edge, a distance from a left inner cuff distal edge to a left outer cuff distal edge, a distance from a left inner cuff proximal edge to a right inner cuff proximal edge, a distance from a left inner cuff proximal edge to a left outer cuff distal edge, a free height of the inner cuff, inner cuff hem fold width, inner cuff elastics length, outer cuff elastics length, core length, and backsheet width.

Further, each of the first and second chassis may comprise identical chemical compositions of one or more of a topsheet, backsheet film, backsheet nonwoven, core super absorbent polymers, core pulp, core nonwoven, core tissue, leg cuff film, leg cuff nonwoven, super absorbent polymer adhesive, core nonwoven adhesive, leg cuff elastic adhesive, and backsheet nonwoven/film adhesive.

And, each of the first and second chassis may comprise the same basis weight of one or more of the topsheet, backsheet film, backsheet nonwoven, core super absorbent polymers, core pulp, leg cuff nonwoven, leg cuff film, super absorbent polymer adhesive, leg cuff adhesive, and backsheet nonwoven/film adhesive.

And, each of the first and second chassis may comprise compositionally identical core super absorbent polymers. The first and second chassis may have identical component cross sectional order and disposition in at least one of the front waist region, back waist region, and crotch region. The inner leg cuffs of the first and second chassis may be composed of the compositionally identical materials.

And, the core adhesives of the first and second chassis may be the same adhesive(s). The first and second chassis may comprise core super absorbent polymers that are in the same chemical class and subclass.

And, each of the first and second chassis may comprise first and second wetness indicators, respectively, and wherein the first and second wetness indicators are compositionally identical.

Further, the inner leg cuffs of the first and second chassis may have identical component cross sectional order and disposition in at least one of the front waist region, back waist region, and crotch region. The distance from the left outer cuff distal edge to a right outer cuff distal edge may the same. The distance from the left inner cuff proximal edge to left outer cuff distal edge may be the same. The distance from the left inner cuff proximal edge to the right inner cuff proximal edge is the same. The lengths of the inner and outer cuffs are the same.

### Test Methods

### Elastic Modulus of the Belt Test

The Elastic Modulus of the Belt is determined by stretching a representative section (referred to as “sample”) of a product's elastic waist belt using a constant of extension tensile testing machine with computer interface, e.g., Instron; MTS; Zwick; etc., using a load cell for which the forces measured are within 10% to 90% of the limit of the cell. The instrument is equipped with single line contact grips wider than the specimen to be tested. The testing is performed under ambient room conditions (temperatures from between 15° C. to 35° C. and relative humidity from between 35% to 75%). Samples are conditioned for at least two hours prior to testing under the same conditions. Linear length measurements for sample preparation are made with a device that is traceable to NIST or other standards organization.
The sample is taken from the back of the product. All layers of the product are included in the sample. The sample is cut using sharp scissors. The sample region is bounded by the rectangle defined between points E, F, H & G on FIG. 3, where the line E-F is 3.0 cm±0.1 cm from the back edge of the product defined by line A-B, and the line G-H is 4.0 cm±0.1 cm from line E-F. The sample is centered in the product, bisected by L1.
The initial gauge length of the tensile testing machine is set at 15.0 cm. The sample is mounted in the grips with minimal slack with the direction of extension corresponding to the lateral direction of the product, and with the center point of the sample centered between the grips. Excess material above and below the upper and lower grip lines, respectively, may be removed to facilitate mounting of the sample. Once the sample is in the machine, the machine's force channel is set to zero (which eliminates the weight of the sample in the calculations). The grips are slowly moved apart at 2.0 in/min (5.08 cm/min) until a load of 0.05 N is attained. The gauge length at this position is recorded as Lo (Lo=15.0 cm+ additional extension to reach 0.05 N).
Extend the sample at a rate of 20 in/min (50.8 cm/min) until either 400 gf is reached or the sample breaks.
The sample initial width (in the product longitudinal direction) for stress calculations is 4.0 cm.
For stress calculations, the thickness of the material is neglected; hence the modulus values are reported in units of force per sample width (gf/mm).
Sample stress is calculated by dividing the force in the machine's load cell by the sample initial width.
Sample strain is calculated by ΔL/Lo.
Record the sample strain at sample stresses of 3 gf/mm; and 7 gf/mm.
The Elastic Modulus of the Belt is the linear slope between these two values (Elastic Modulus of the Belt=[7 gf/mm−3 gf/mm]/[sample strain at 7 gf/mm− sample strain at 3 gf/mm]). Units are gf/mm of width.
10 products are tested and the Elastic Modulus is the average.

### Array Elastic Modulus of the Belt

The Array Elastic Modulus of the Belt is the average Elastic Modulus of the Belt for each size offered in a product array. For example, the Depend Underwear for Women (Maximum Absorbency) is marketed and sold in an array of 3 sizes: Small/Meduim; Large and Extra Large. The Array Elastic Modulus of the Belt is the average of: The Elastic Modulus of the Belt for size Small/Medium; the Elastic Modulus of the Belt for size Large; and the Elastic Modulus of the Belt for size Extra Large. Table 3 shows examples of the Array Elastic Modulus of the Belt for some currently marketed product arrays.

### Relaxed Waist Circumference

The relaxed waist circumference for each size in the array is determined by:
Cut out a full circumferential section of the products waist belt (referred to as “waist hoop”). The testing is performed under ambient room conditions (temperatures from between 15° C. to 35° C. and relative humidity from between 35% to 75%). Samples are conditioned for at least two hours prior to testing under the same conditions. Linear length measurements for sample preparation are made with a device that is traceable to NIST or other standards organization.
The waist hoop is defined by the rectangular section: E, F, G & H at the back of the product, where E-F is 3.0 cm±0.1 cm from the back edge of the product defined by A-B, and G-H is 4.0 cm±0.1 cm from E-F. At the front of the product, the waist hoop is continued by adding the material defined by the rectangular section: E′, F′, G′ & H′, where E′-F′ is 3.0 cm±0.1 cm from the front edge of the product defined by A′-B′, and G′-H′ is 4.0 cm±0.1 cm from E′-F′.
For a closed form product (continuous waist hoop), cut the waist hoop (E, F, G, H, E′, F′, G′ & H′) at one of the side seams (A-D/A′-D′ or B-C/B′-C′) creating a single strip of waist hoop. Allow this single strip to hang vertically with a load of 5.0 gf at the end.
The measured length of this strip is the relaxed waist circumference for this size in this array.
For an open form product, the relaxed waist circumference is the summation of sections E-F-G-H and E′-F′-G′-H′ (each strip vertically hung with a load of 5.0 gf at the end).

The dimensions and values disclosed herein are not to be understood as being strictly limited to the exact numeral values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An array of packages comprising three or more different sizes of absorbent articles, the array comprising:
a first package comprising a first absorbent article having a first size;
a second package comprising a second absorbent article having a second size;
a third package comprising a third absorbent article having a third size;
wherein the Array Elastic Modulus of the Belt of the first, second, and third package is from about 6 gf/mm to about 15 gf/mm; and
wherein the first, second, and third packages comprise the same brand name and sub-brand name.

2. The array of packages of claim 1, wherein the first absorbent article comprises strands in the waist region and the second absorbent article comprises an elastomeric film in the waist region.

3. The array of packages of claim 1, wherein at least one of the packages have an Elastic Modulus of the Belt from about 2 gf/mm to about 10 gf/mm.

4. The array of packages of claim 1, wherein at least one of the packages have an Elastic Modulus of the Belt from about 3 gf/mm to about 8 gf/mm.

5. The array of packages of claim 1, wherein at least one of the packages have an Elastic Modulus of the Belt from about 3 gf/mm to about 7 gf/mm.

6. The array of packages of claim 1, wherein at least one of the packages have an Elastic Modulus of the Belt from about 4 gf/mm to about 6 gf/mm.

7. The array of packages of claim 1, wherein at least two of the packages have an Elastic Modulus of the Belt from about 3 gf/mm to about 7 gf/mm.

8. The array of packages of claim 1, wherein each of the absorbent articles comprises a pair of permanent side seams.

9. The array of packages of claim 1, wherein each of the absorbent articles comprises a pair of refastenable side seams.

10. The array of packages of claim 9, wherein the refastenable side seams are formed by hooks and loops.

11. The array of packages of claim 1, wherein the first absorbent article comprises strands in the waist region and the second absorbent article comprises an elastomeric film in the waist region.

12. The array of packages of claim 1, wherein the first and second articles have Elastic Modulus of the Belt values greater than 5 gf/mm apart.

13. The array of packages of claim 1, wherein the first and second absorbent articles comprise compositionally identical core super absorbent polymers.

14. The array of packages of claim 1, wherein the first and second absorbent articles of the first and second packages comprise chassis having identical cross-sectional dispositions in at least one of a front, back, or crotch region.

15. The array of packages of claim 1, wherein the array of packages is an on-line array.

16. The array of packages of claim 1, wherein the at least two packages comprise adult incontinence disposable absorbent articles in closed form.

17. An array of packages consisting of two different sizes of absorbent articles, the array comprising:
a first package comprising a first absorbent article having a first size;
a second package comprising a second absorbent article having a second size;
wherein the Array Elastic Modulus of the Belt of the first and second package is from about 2 gf/mm to about 5 gf/mm; and
wherein at least two of the first and second absorbent articles of the first and second packages comprise substantially identical chassis.

18. An array of packages comprising three or more different sizes of absorbent articles, the array comprising:
a first package comprising a first absorbent article having a first size;
a second package comprising a second absorbent article having a second size;
a third package comprising a third absorbent article having a third size;
wherein the Array Elastic Modulus of the Belt of the first, second, and third package is from about 10 gf/mm to about 15 gf/mm; and
wherein the first, second, and third packages comprise the same brand name and sub-brand name.

19. The array of packages of claim 18, wherein the first and second absorbent articles comprise compositionally identical core super absorbent polymers and compositionally identical core adhesives.

20. The array of packages of claim 19, wherein the first absorbent article comprises a first chassis and the second absorbent article comprises a second chassis, wherein inner leg cuffs of the first and second chassis may have identical component cross sectional order and disposition in at least one of a front waist region, back waist region, and crotch region.
